(19) United States
(12) Patent Application Publication
(10) Pub. No.: US 2010/0068345 A1
(43) Pub. Date: Mar. 18, 2010

(54) Method for Production of Individual Milk Products

(75) Inventors: Talo Willem Tamminga, Cambridge (CA); Alexander Van Der Lely, Rotterdam (NL)

Correspondence Address: HOWREY LLP-EU, C/O IP DOCKETING DEPARTMENT, 2941 FAIRVIEW PARK DR., SUITE 200, FALLS CHURCH, VA, 22042, US

(73) Assignee: LANVI S.A.R.L., MAASSLUIS (NL)

(21) Appl. No.: 12/516,395

(22) PCT Filed: Nov. 26, 2007
(86) PCT No.: PCT/NL07/00293

§ 371 Date: May 27, 2009

Related U.S. Application Data
(63) Continuation of application No. 11/604,369, filed on Nov. 27, 2006.

(51) Int. Cl. A23C 9/00; A01J 5/007; A23C 9/158; G01N 33/04; B25J 11/00

(52) U.S. Cl. 426/72; 119/14.02; 119/14.08; 426/87; 901/50

(57) ABSTRACT

A system and method for producing milk is disclosed in which the milk from a single animal is handled and processed separately from milk produced by other animals. On packaging the processed product, an indication specific to that animal may be included on the package.

### BACKGROUND OF THE INVENTION

1. Field of the Invention

The invention relates generally to milk production and processing methods and more particularly to methods of producing milk products that provide improved identification of the dairy animal from which the milk has originated. The invention further relates to a system for processing milk and an improved milk product produced according to such methods.

2. Description of the Related Art

It is well known to produce milk by milking cows or other dairy animals using milking machines. Today, relatively little milk is produced by hand and this is of an insignificant commercial value and will not be discussed further. Most milking herds are milked twice or three times a day. This may take place in a dedicated milking parlour whereby the cows are led or driven to the milking parlour for milking and then released. The cows may be kept indoors in cowsheds or may be kept outdoors and brought to the parlour at milking time. This may vary from country to country and also according to the time of year.

An alternative form of milking arrangement is the tie stall with pipeline. In such a system, the cows may be tethered and milked at their feeding station. Pipelines extend throughout the cowshed to provide milking vacuum to and transport milk from each milking device. The cows are then milked according to the particular milking scheme implemented by the farmer or dairyperson. In certain tie stall cowsheds, the animals may be let out periodically to pasture.

Milk produced according to the above milking procedures is usually collected in bulk tanks at the milking facility. The milk from all animals in a herd will thus be mixed, with the exception of milk that is found to be defective. This may be determined by testing the milk for the presence of contaminants and separating such milk for disposal or for alternative uses. Milk from certain animals may also be excluded on the basis of data relating to the animal e.g. lactation stage, sickness etc. Periodically, milk will be collected from the facility by a bulk tanker that transports the milk to a processing facility or dairy. Here the milk may be further mixed with milk from other facilities and the provenance of the milk in the finally packaged product is usually lost. According to food authority requirements, the packaged product will generally be marked with an indication of its composition as determined at the processing and packaging plant.

Recent trends have encouraged the development of organic initiatives and the like in which milk produced according to certain criteria is produced and marketed separately. The product packaging usually includes a reference to the criteria e.g. “organic milk” by which a consumer may be assured that the product has met the required standards. It is also not uncommon for dairy produce to include an indication of the milking facility from which the milk originates. This may be achieved by processing directly at the milking facility or by separate batch processing at a dedicated processing facility. Increasingly, there is a desire to provide more information regarding the exact origin of purchased dairy produce. This information is of interest both to consumers and to regulatory authorities.

Robotic milking machines have also been developed that provide increasingly sophisticated monitoring possibilities. These milking robots allow the teat cups of the milking machine to be connected to the teats of the cow or other dairy animal automatically. The whole milking procedure may thus be carried out without intervention of a human operator. Use of a robot thus allows the animal to choose when it wishes to be milked. Since a human operator is absent, there is no longer a need to adhere to a specific time table. Each cow may individually decide how frequently she wishes to be milked. For some cows this will be twice a day, for other cows three times. Such milking has been shown to improve the yield of an animal and significantly avoids problems due e.g. to over distended udders.

By allowing the cows to report for milking on a voluntary basis, the milking machine may be used more effectively. A single milking robot may serve to milk a herd of around 60 animals. The robot may therefore include further refinements and procedures that would perhaps be too expensive were they to be replicated in a multiple stall milking parlour. Furthermore, since a human operator is not present, monitoring of the animals health and the milk quality must be performed by the robot. Since robots are presently better suited for monitoring all possible faults at an early stage, the resulting standard of care may be significantly improved and extremely low levels of bacteria and somatic cells are frequent in milk produced using today's robots.

Robot systems that are available at present include the Astronaut™ milking system available from Lely International and the VMS™ milking system available from DeLaval. Although robotic milking machines can collect and monitor the milk from an individual animal, the milk is subsequently passed to a bulk tank. Thereafter it is collected and processed together with milk from other sources. Any specific characteristics of this milk are therefore diluted and cannot be referenced during the packaging of the milk.

A device is known from EP-A-0628244 in which a robotic milking device is used for separating milk into different containers according to quality or composition. Various factors may be used for the separation. In particular, it may use appropriate sensors to determine the somatic cell count of the milk that could be indicative of contamination. Alternatively, the milk may be separated according to the individual animal, fat content, albumin content, colour or lactation stage. Another arrangement is suggested in EP-A-1369030 in which the milk from certain animals may be at least partially separated. By carefully monitoring the milk production of each animal, the total production of the herd may be controlled and maintained between certain criteria. This is described as useful in meeting milk production quotas in relation to quantity and composition of milk. In a further known device, a number of milking robots supply milk from the herd directly to a cheese production facility. The cheese produced is thus directly related to the particular herd. Although these devices provide a limited ability to separate the milk and influence the composition of the finally packaged product, they do not provide for the possibility of relating the final product to an individual animal.

### BRIEF SUMMARY OF THE INVENTION

The present invention addresses these problems by providing an improved method of producing milk. The method comprises: providing a milking machine located at a milking facility; allowing an individual dairy animal access to the milking machine for the purpose of milking; milking the animal to produce milk; determining cow specific data related to the milk produced; processing and packaging the milk to form a processed milk product; and marking the processed milk product with an indication of the cow specific data. The packaged milk product may then be subsequently transported and marketed as required. By separately and exclusively processing the milk produced by a given animal, it is also possible to indicate and take advantage of its improved characteristics. Such characteristics may include, improved fat, protein or vitamin content and lower levels of impurities and microbial contamination. In the present context cow specific data is understood to mean data that is specifically related to the individual cow or the milk produced by that cow. It is also noted that reference in the following to cow is understood to also include goats, buffalos and any other appropriate dairy animal.

According to an important aspect of the invention, the milking machines are milking robots that automatically perform the milking of the dairy animals. In this manner, greater efficiency of the complete milk processing chain may be achieved and human intervention is minimised. It is a characteristic of milking robots that a single robot may be used for a herd of around 60 animals and that it is sequentially used throughout the day (and night). Conventional milking parlours involve the simultaneous milking of e.g. 6, 8 or 12 animals. In such a case, there is thus directly a greater complexity in maintaining and processing separate milk streams. While a single robot may be used with a dedicated packaging line, it is also understood that in bigger facilities, a number of robots may at least partially feed into a single line.

According to one aspect of the invention, the package may be marked with the identity of the animal. This may comprise the name of the cow and details of the milking facility. Alternatively, it may merely comprise data for registration purposes that uniquely identifies the animal. Another possibility is that a picture or photograph of the actual cow is included on the package. This may be based on a historical record or may be taken during the visit of the animal to the milking machine.

Additionally or alternatively, the data included on the package may include milk quality data including, but not limited to: actual composition; fat content; protein content; butyric acid content; freezing point; free fatty acid value; melatonine content; and vitamin content. The data may also include details for regulatory purposes such as levels of noxious substances including pesticides, dioxins and heavy metals; hormone content and the like. Since the data is derived for the individual animal in relation to a given milking, the information provided on the package actually relates to the product inside rather than to a herd or production facility average.

Data tested and recorded may also include details relating to the animal condition including but not limited to combinations of any of the following: variations from its normal rhythm or variable milking periods, excessive movement in the stall, body score, and the time spent indoors or in the pasture, details of distances moved e.g. in the pasture using GPS or with a step counter, sleep details, rumination details, urine and faecal analysis, animal breed, time or day at which milking takes place, lactation stage, climatic conditions and the like. The above data may be stored in an appropriate database and used in formulating and determining a general score for the condition of the animal. Certain selected information may be marked directly onto the packaging e.g. the distance walked today. Other data may be used for indicating compliance with e.g. organic criteria or for separating or targeted processing of the milk. Alternatively the data may be used merely for monitoring purposes in order to follow or predict cow behaviour and welfare.

Of particular importance is the monitoring of feed consumption and details of an animal's particular diet including details of the pasture This may be important for monitoring purposes but it is also of great interest to the consumer to be able to read on the package information regarding the feed provided to the animal that produced the milk. This marked data may be general e.g. “fed with only organic produce” or may be specific. The data may also be used for selection of the milk or for fine tuning of the milk processing. It is thus conceivable that all milk produced from cows that had been in the pasture would be treated and packaged separately. This could be determined by observing the fact that the cow had moved a given distance to graze. Furthermore, certain animals could be given special feed or supplements and their milk could then be processed separately. Thus although the invention may primarily relate to packaging of milk from a single animal, it is also considered to encompass the selective processing of milk from a group of animals that meet a certain criteria. In this manner, e.g. milk collected at night could be collected and processed separately with an indication of this fact and other details related to the qualities of milk taken at night.

Consumers are increasingly conscious of ethical and environmental aspects related to farming. Providing and guaranteeing that dairy products have been produced according to accepted criteria has an added advantage in the marketing of such products. In this manner, individual consumers have the opportunity to influence the manner in which animals are treated according to their purchase patterns. Accordingly, the complete process including milking, transport, processing, packaging and distribution facilities may also be tested according to environmental criteria. Stringent environmental criteria may be defined e.g. by a coordinating organization responsible for the standard. These may include criteria relating to farming and husbandry such as the care for the land and the use of environmentally acceptable products and feeds. They may also relate to the modes of transport and the fuels used and the energy efficiency and design of the processing facility or the sort of milking machine used. In relation to the packaging and marketing of the dairy produce, environmental criteria may dictate the nature of the packaging used, requiring it to be recyclable or reusable. As above, this data may be stored in a database and used in formulating and determining compliance with criteria or e.g. for separating or targeted processing of the milk.

According to a preferred aspect of the present invention, the milking machines may be present at a single milking facility and the processing and packaging may take place at least partially at the milking facility. This may also lead to advantageous improvements in the overall efficiency of the process and to higher quality products and less wastage. Such process steps may include cooling, sorting, testing, grading, separating, sterilizing, homogenising, pasteurizing, mixing, supplementing with additives and vitamins and the like. Further data related to this processing may also be recorded and maintained in a database and may be marked on the package e.g. together with the data mentioned above. It is nevertheless also envisaged that processing or packaging may take place at least partially elsewhere. In which case, the milk from an individual animal together with its data must be transported separately to the processing facility. This may be achieved using e.g. labelled or tagged milk capsules having a capacity sufficient to receive the milk of a single animal. The capsule could also be sufficiently large to keep the milk of an animal separate from a number of milkings.

The invention also relates to a system for processing milk comprising a milking machine to which dairy animals may have access for the purpose of milking; sensors for determining cow specific data related to the milk produced; a processing facility for converting the milk into a processed milk product; and a marking arrangement for marking the processed milk product with an indication of the cow specific data. Such a system may provide the benefits of the method as described above.

According to a desirable feature of the invention, the processing facility or the marking arrangement is operable to receive and maintain information relating to the milk being collected or processed. In particular all or any data registered and stored by the milking facility may be transmitted or otherwise provided to the processing facility. This may be extracted from a database and may take the form of a database record or token relating to a batch of milk that is passed from the milking machine to the processing or packaging facility. The data may be carried on electronic chips or in the form of labels or the like, which can be affixed to the product at a later stage. Alternatively, the packaged milk product may be merely marked or tagged and the data corresponding to this product may be kept separately for consultation if required.

Most preferably, the system also includes a device or other means for separating the milk such that only milk meeting a given criteria is processed and marked in this way. The remainder of the milk is processed conventionally.

The invention also relates to a processed and packaged dairy product derived exclusively from the milk of a single dairy animal, the product being marked with an indication of animal specific data relating to the single dairy animal. The animal specific data may be any of the data as provided above and may be printed onto the package or provided as a separate label or chip. The package may be any conventional package appropriate to the product. In particular for milk, sealed cartons or bottles may be preferred. Most preferably, the package includes a picture of the animal. In this manner, a consumer entering a store will be presented with a plurality of packages, each containing product from a single dairy animal and having different pictures and information related to the animals.

According to a further aspect, the invention also relates to a device for taking portrait style photographs of dairy animals. The device comprises an exit gate from a milking facility provided with a camera and an identification system. A photograph taken as the animal exits the facility may then be associated with data for that animal and may include the name of the milking facility. The device may be provided and used in combination with a cleaning arrangement to ensure that the animal is well groomed.

### BRIEF DESCRIPTION OF THE DRAWINGS

The features and advantages of the invention will be appreciated upon reference to the following drawings, in which:

FIG. 1 is a schematic view of an embodiment of the invention showing the various facilities involved in the production of a dairy product;

FIG. 2 is an elevation of an alternative milking robot according to a second embodiment of the invention for use in the milk processing system of FIG. 1; and

FIG. 3 is a view of an exit gate arrangement from a cowshed.

DESCRIPTION OF ILLUSTRATIVE EMBODIMENTS

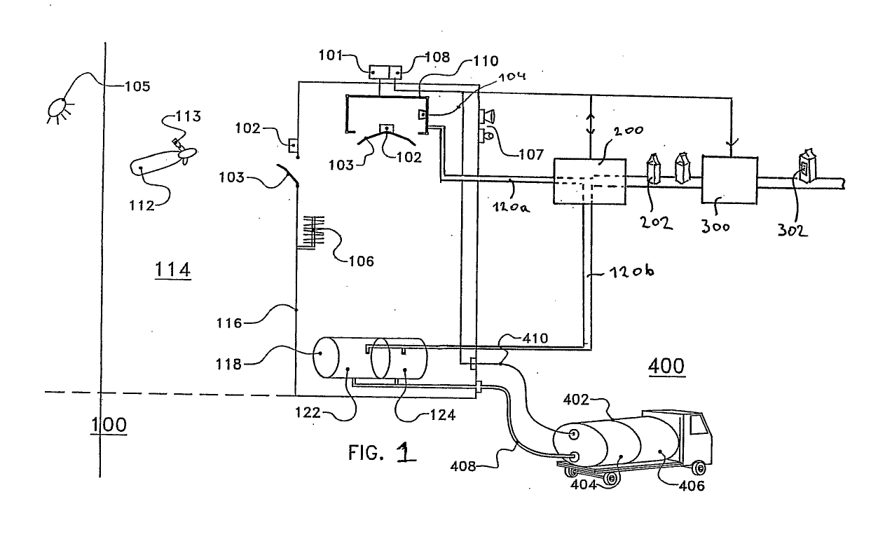

The following is a description of certain embodiments of the invention, given by way of example only and with reference to the drawing. Referring to FIG. 1, a milk processing system 1 according to a first aspect of the invention is shown comprising a milking facility 100 in which is located a milking robot 110. For the present purpose, the milking facility 100 may be considered to be an independently operating farm. While one milking robot 110 has been shown, it is also possible that a milking facility has a plurality of milking robots 110, depending on the size of the herd. Milking robot 110 is preferably of the Astronaut A3™ type, available from Lely and will not be further described at present. It is nevertheless understood that alternative milking devices may also be used, subject to the criteria defined below. They milking robot 110 is provided with appropriate sensors as well known to the skilled person and e.g. as described in EP-A-628244, the contents of which are incorporated herein by reference in their entirety. A characteristic of the milking robot 110 is that it allows voluntary milking as and when an individual animal demands.

Cows 112 are free to graze in the pasture 114 of the milking facility 100 and are also able to take shelter in cowshed 116 at night or during inclement weather. For convenience and environmental protection, the milking robot 110 is also located in cowshed 116. It is however understood that the robot 110 may also be located externally in the pasture 114 in order to provide more immediate access for the cows 112 during grazing. Also located in cowshed 116 is a milk tank 118 for collecting, storing and cooling milk. The milk tank 118 is connected to the milking robot 110 by milk lines 120.

Each cow carries an identity device 113. The identity device 113 also serves as a GPS positioning beacon allowing location of the cow 112 within the pasture 114 and tracking of its movements. Within the cowshed 116, milk tank 118 comprises a first milk section 122 and a further milk section 124. It will be understood that sections 122, 124 may alternatively be embodied as separate tanks and that separate milk lines may be provided for each.

The milking facility 100 is provided with a computer 101 which controls operation of the milking facility 100. The computer 101 controls the milking robot 110 and its sensors and may also interact and/or control other features of the milking facility 100 including but not limited to: identification systems 102, gates 103, feeding arrangements 104, GPS positioning systems 105, cleaning arrangements 106 and signalling arrangements 107. The computer 101 includes a database 108 in which all relevant data related to the operation of the milking facility 110 is stored. In particular, for each individual cow 112, the database 108 comprises a record containing data including but not limited to: animal identity; milk quantity; milk quality and composition including milk fat content and free fatty acid value, milk protein content, lactic acid content, butyric acid content, freezing point, milk colour, somatic cell count, bacterial count, levels of noxious substances such as pesticides, dioxins and heavy metals, hormone content, melatonin content and vitamin content; animal condition including variations from its normal rhythm or variable milking periods, excessive movement in the stall, body condition score, feed consumption and details of its particular diet, details of the pasture and the time spent indoors or in the pasture, lactation stage, sleep details, rumination details, urine and faecal analysis; animal breed; time or day at which milking takes place; and climatic conditions.

The database 108 is able to retrieve and update data relating to an individual cow 112 based on its identity device 113. Movements of the animal in the pasture 114 may be fed back to the computer 101 and database 108 via GPS system 105. Approach of an animal to a gate 103 may be detected by identification system 102 and the gate opened if required. Feeding arrangement 104 may be actuated by computer 101 in response to arrival of a given cow 112 at the robot 110 for milking. Unfinished feed may be detected and recorded in the database 108. Milk from the robot 110 may be selectively directed to either section 122 or 124 according to the identity of the cow or any other criteria related to milk quality or as further defined herein. The skilled person will immediately appreciate that many further possible details can be measured and recorded as desired by the farmer or authorities and that appropriate sensors and systems may be included accordingly.

Also schematically shown in FIG. 1 are a processing facility 200 and a marking facility 300. Processing facility 200 is a selective dairy produce processing facility and is connected in the milk line 120 between the robot 110 and the milk tank 118. The processing facility 200 processes some of the milk received from the milking robot 110 into a packaged dairy product 202. The product 202 is conveyed to the marking facility 300 where it is marked with a label 302 carrying animal specific data. The processing facility 200, the marking facility and the milk tank 118 are all connected to the computer 101 via a data bus 190.

FIG. 1 also indicates a milk transporting facility 400. Milk transporting facility 400 comprises a bulk tanker 402, adapted to receive milk from the milk tank 118 and transport it for processing at an external facility. The bulk tanker 402 also comprises two separate milk compartments designated as first milk compartment 404 and further milk compartment 406. The bulk tanker 402 is connected to the milk tanks by a pipe 408. According to the milk collected and its compliance with chosen criteria, the milk will be delivered through the pipe 408 to one of the compartments 404, 406. Under normal circumstances, milk from section 122 will be delivered to compartment 404 and milk from section 124 will be delivered to compartment 406. Although not shown, it is understood that the pipe 408 may be provided with a number of channels for keeping the different milk supplies separate. In addition to carrying milk, pipe 408 provides a data connection 410 from the computer 101 and database 108 to the bulk tanker 402 over data bus 190. In this manner, data relating to the milk may also be transferred to the bulk tanker where it is maintained on an appropriate data carrier (not shown). It is also envisaged that alternative milk transporting systems could be employed which would be equivalent to or instead of the bulk tanker depicted e.g. based on the use of milk cans. Such milk cans could be provided with data in the form of electronically readable tokens, bar codes, labels or the like.

In use, the system 1 works as follows. The cows 112 graze in the pastures 114 and are able to report voluntarily to the milking robot 110 at any point in time when they desire to be milked. Arrival of a cow 112 at the milking robot 110 is detected by identification system 102 recognising identification device 113 and the milking procedure may commence. The milking procedure may comprise a number of steps including grooming, washing, pre-milking, milking, post milking, disinfecting, feeding and any other procedures that it may be appropriate to perform on the animal in question. Although generally speaking a cow 112 may report to be milked at any moment that she desires, there may nevertheless be placed limits on the number of times she may present herself within a given period. These measures are well known to the skilled practitioner in the field of milking robots and will not be further dealt with here. The milk collected by the milking robot 110 is passed via the milk line 120a to the processing facility 200. Data related to the milk and the individual animal that provided the milk is also passed over the data bus 190 to the processing facility. Based on the data, the processing facility will decide whether the milk should be immediately processed or not. This decision may be taken based on the quality of the milk, the identity of the animal, the feed ingested by the animal of the like. In the present example, milk is selected based on the following criteria:

a. Fat content between 3.5 and 4.5%

b. Protein content greater than 3.0%

c. Lactose content between 4.2 and 4.8%

d. Somatic cell count below 100 000.

e Bacterial count below 5 000.

f. Cow not sick

If the milk is not to be immediately processed, it continues via milk line 120b to the milk tank 118, where it is stored for subsequent collection by milk tanker 402. This milk will be processed by conventional procedures at a separate milking facility. Nevertheless, by the use of separate milk sections 122, 124 the milk can still be graded such that certain qualities or types of milk may receive separate treatment. Control of the milk tank to determine which section receives a given quantity of milk takes place over the data bus 190. It will of course be understood that still further milk may be entirely discarded e.g. at the milking robot, if it is deemed unsuitable for human consumption.

The milk processed by the processing facility 200 is subjected to a number of process steps. In the present example, the process step is limited to the step of packaging. It will however be immediately evident to the skilled person that other steps may be carried out. These may include cooling, sorting, testing, grading, separating, sterilizing, homogenising, pasteurizing, mixing, supplementing with additives and vitamins and are not further discussed in detail here. It is also possible that further process steps are included to change the nature of the milk for the production of cheese, butter, yoghurt or ice-cream. The milk is dispensed into one litre cartons as conventionally used in many countries for the marketing of milk. Since a cow will never produce a consistent quantity of milk corresponding to a number of complete litres, for each animal attaining the above criteria, five litres of milk is taken for packaging at the processing facility 200. The remainder is sent to the milk tank 118.

The processed and packaged dairy product 202 exits the processing facility and is conveyed to the marking facility 300. Here it receives a label 302 indicating the animal specific data relating to its content. The label may be printed by a conventional label printer on the basis of data received over the data bus 190. In particular, the label includes a picture of the individual cow as it leaves the milking robot together with its name.

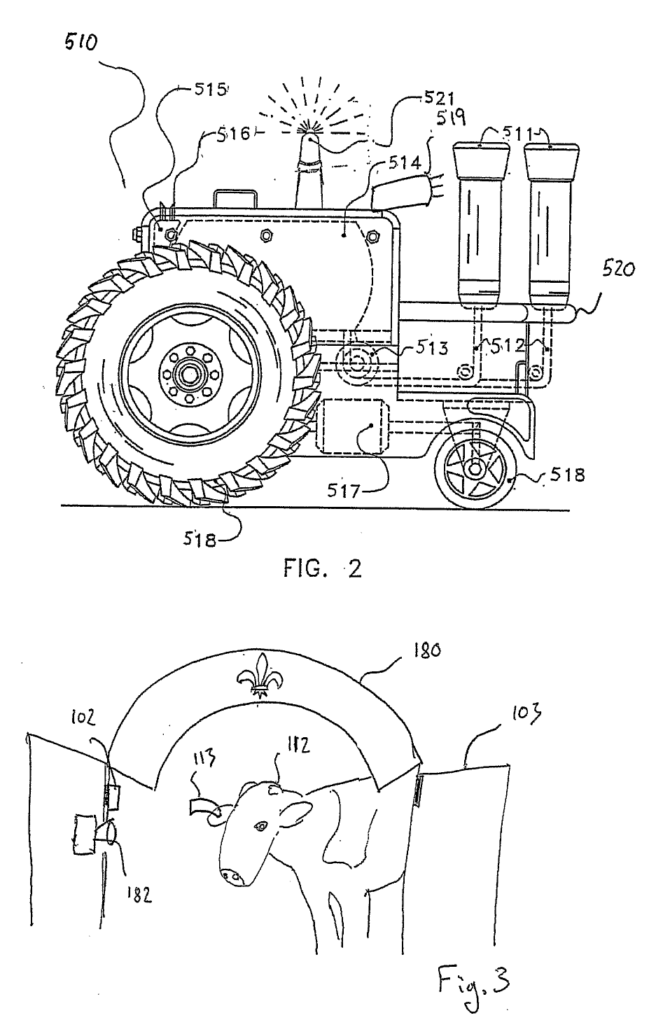

FIG. 2 shows an alternative arrangement of a milking robot 510 that may be used instead of the robot 110 of FIG. 1. Robot 510 is a self propelled autonomous device of the sort generally described in US2002/033137, the contents of which are incorporated herein by reference in their entirety. Robot 510 comprises teat cups 511 for connection to the teats of an animal and milk tubes 512 and vacuum pump 513 for extracting the milk and delivering it to a milk capsule 514. The robot 510 receives power from a battery 515 that may be connected to a mains supply for recharging via a socket 516. As well as powering the vacuum pump 513, the battery also powers a motor 517 for turning driving wheels 518. Steering wheels 518 are provided for guiding the robot 510 on the basis of signals received by a signalling device 521. It is understood that signalling device 521 may comprise a GPS positioning device or another form of positioning device. Signalling device is also adapted to receive information regarding the identity of an animal from its identity device 113. A laser detector device 519 is used to locate and identify the teats of the animal and lift 520 raises the teat cup 511 for application to the appropriate teat.

According to the invention, capsule 514 is detachable from the robot 510. In this manner, once milking has been completed, the milking robot 510 may report to processing facility 200 where the (partially) filled capsule 514 is released and replaced by an empty capsule 514. The filled capsule 514 is processed within the processing facility 200 as described above. Data may be transmitted to the processing facility 200 together with the capsule 514, instead of via a data bus.

FIG. 3 shows another aspect of the invention including an exit gate 103 from a cowshed 116. The exit gate 103 includes an eye-catching or otherwise appropriate sign 180 that may include the name of the milking facility. Also positioned in front of the gate is a camera 182. On leaving the cowshed 116, the cow is identified by identification system 102 and identity tag 113. In response to this identification, the gate 1003 opens and as the animal 112 exits, a portrait style photograph is taken by the camera 182. This photograph is placed onto the data bus 190 and may be stored by database 108. In order to ensure that the animal 112 looks its best and is generally free of dirt, flies and the like, it may first be caused to pass cleaning arrangement 106. The exit gate 103 may also be an exit gate from a milking robot 110 or alternatively could form part of the exit gate from a milking roundabout as generally described in US2005/0076838, the content of which is also incorporated herein by reference in its entirety.

Thus, the invention has been described by reference to the embodiments discussed above. It will be recognized that these embodiments are merely exemplary and are not limiting upon the scope of the invention. Alternative or additional criteria may be used for sorting and separating the milk to be processed. Further data in addition to that described above may be collected and processed for identifying and qualifying the milk. Many modifications in addition to those described above may be made to the structures and techniques described herein without departing from the spirit and scope of the invention.

What is claimed is:

1. A method of producing milk products, comprising:
providing a milking machine located at a milking facility;
allowing an individual dairy animal access to the milking machine for the purpose of milking;
milking the animal to produce milk;
determining cow specific data related to the milk produced;
processing and packaging the milk to form a processed milk product; and
marking the processed milk product with an indication of the cow specific data.

2. The method of claim 1, wherein the milking machine is a milking robot that automatically performs the milking of the dairy animal.

3. The method according to claim 1 or claim 2, wherein the data comprises information related to the identity of the animal.

4. The method according to any preceding claim, wherein the data comprises information related to the quality of the milk produced.

5. The method according to any preceding claim, wherein the data comprises
information related to the condition of the animal.

6. The method according to any preceding claim, wherein the data comprises
information related to the feed received by the animal.

7. The method according to any preceding claim, wherein the data comprises information related to the conditions under which the milk produced.

8. The method according to any preceding claim, wherein the processed milk product is marked with further data related to the manner in which the milk is processed.

9. The method according to any preceding claim, wherein the production, processing and packaging of the milk are performed in a substantially continuous process at the milking facility.

10. The method according to any preceding claim, wherein the processed milk product comprises milk from only the individual animal.

11. The method according to claim 10, wherein the processed milk product comprises milk from a plurality of milkings of the individual animal.

12. The method according to any preceding claim, wherein data or the further data is tested against certain criteria and only milk for which the criteria is met is processed and packaged to form the processed milk product.

13. The method according to claim 12, wherein the criteria are selected from the group consisting of: animal identity; milk quantity; milk quality and composition including milk fat content and free fatty acid value, milk protein content, lactic acid content, butyric acid content, freezing point, milk colour, somatic cell count, bacterial count, levels of noxious substances such as pesticides, dioxins and heavy metals, hormone content, melatonin content and vitamin content; animal condition including variations from its normal rhythm or variable milking periods, excessive movement in the stall, body condition score, feed consumption and details of its particular diet, details of the pasture and the time spent indoors or in the pasture, lactation stage, sleep details, rumination details, urine and faecal analysis; animal breed; time or day at which milking takes place; and climatic conditions.

14. A system for processing milk comprising:
a milking machine to which a dairy animal may have access for the purpose of milking;
sensors for determining cow specific data related to the milk produced by an individual animal;
a processing facility for converting the milk into a processed milk product; and
a marking arrangement for marking the processed milk product with an indication of the cow specific data.

15. The system of claim 14, wherein the marking facility applies a label to the product.

16. The system of claim 14, wherein the marking facility prints a marking onto the product.

17. The system of any of claims 14 to 16, further comprising a computer including selection criteria and a further processing facility, the computer selecting the milk on the basis of the selection criteria and directing it to either the processing facility or the further processing facility.

18. The system of claim 17, wherein the further processing facility comprises a milk tank in which the milk from a plurality of animals is received.

19. The system of any of claims 14 to 18, wherein the milking machine comprises an automatic milking robot.

20. The system of claim 19, wherein the milking robot is an autonomous milking robot having a milk receptacle for receipt of the milk from a single animal.

21. The system of any of claims 14 to 20, wherein the processing facility is located at a milking facility and is connectable to the milking machine for receipt of milk therefrom.

22. A processed milk product derived exclusively from the milk of a single dairy animal including a package comprising an indication of animal specific data related to the identity of the animal and the milk.

23. The product of claim 22, wherein the indication comprises a photograph of the animal that produced the milk.

24. The product of claim 22 or claim 23, wherein the animal specific data is selected from the group consisting of: animal identity, milk quality, milk composition, milk fat content, milk free fatty acid value, milk protein content, milk lactic acid content, milk butyric acid content, milk freezing point, milk colour, milk somatic cell count, milk bacterial count, levels of noxious substances such as pesticides, dioxins and heavy metals in the milk, milk hormone content, milk melatonin content, milk vitamin content; animal condition, body condition score, feed consumption, diet details, pasture details, time spent indoors or in pasture, lactation stage, sleep details, rumination details, urine and faecal analysis, animal breed, time or day at which milking takes place, climatic conditions, identity of milking facility and type of milking equipment.

25. The product of any of claims 22 to 24, comprising milk that has been subjected to a process selected from: cooling, sorting, testing, grading, separating, sterilizing, homogenising, pasteurizing, mixing and supplementing with additives and vitamins.
